(19) United States
(12) Patent Application Publication
(10) Pub. No.: US 2010/0217129 A1
(43) Pub. Date: Aug. 26, 2010

(54) ANGIOGENESIS MONITORING USING IN VIVO HYPERSPECTRAL RADIOMETRIC IMAGING

(76) Inventors: Wafik S. El-Deiry, Bryn Mawr, PA (US); Paul Tumeh, Philadelphia, PA (US); Jeremy M. Lerner, Hillsborough, NJ (US)

Correspondence Address: Pearl Cohen Zedek Latzer, LLP, 1500 Broadway, 12th Floor, New York, NY, 10036, US

(21) Appl. No.: 12/532,774

(22) PCT Filed: Mar. 20, 2008
(86) PCT No.: PCT/US08/03678

§ 371 Date: Apr. 19, 2010

Related U.S. Application Data
(60) Provisional application No. 60/907,165, filed on Mar. 23, 2007.

(51) Int. Cl. A61B 6/00

(52) U.S. Cl. 600/476

(57) ABSTRACT

This invention relates to the use of in-vivo hyperspectral imaging to monitor angiogenesis. Specifically, the invention provides systems and methods of obtaining hyperspectral images of a field of view comprising an area sought to be monitored.

### GOVERNMENT INTEREST

This invention was supported, in part, by Grant Number T32 NS043126-03 from the NIH; and Grant Number U54105008 from the National Cancer Institute's Network for Translational Research in Optical Imaging (NTROI). The government may have certain rights in the invention.

### FIELD OF INVENTION

This invention is directed to the use of in-vivo hyperspectral imaging to monitor angiogenesis. Specifically, the invention provides systems and methods of obtaining hyperspectral images of a field of view comprising an area sought to be monitored.

### BACKGROUND OF THE INVENTION

Although traditional medical imaging modalities such as computed tomography and magnetic resonance imaging have significantly advanced our understanding of cancer behavior and have provided a means for objectively determining tumor response to anticancer therapeutics, historical definitions of tumor response such as the WHO criteria, varied greatly among researchers, creating a serious challenge in reporting results in a consistent manner.

In an attempt to address these limitations, an international collaboration of academicians, industry representatives, and regulatory authorities established a unified, international standard for tumor response assessment in 2000, termed the Response Evaluation Criteria in Solid Tumors (RECIST), that was based on CT and MRI linear measurements of lesion size. The National Cancer Institute (NCI) has called for the improvement of the RECIST methodology since these anatomical imaging techniques have proved to be less than optimal predictors of therapeutic response in comparison to the detection of molecular events that may better correlate with diagnosis, staging, prognosis and response to cancer therapy.

Classical hyperspectral imaging instruments are based on changing filters using a liquid crystal tunable filter LCTF, Acousto Optic Tunable filter (AOTF) or an interferometer. The consequence is that these devices cannot be used if the objects in the field of view (FOV) move or change during image/data acquisition. Likewise, classical wavelength dispersive instruments use diffraction gratings with poor light throughput and overlapping spectral orders.

Therefore there is a need for an objective and reproducible, accurate and sensitive method of monitoring and imaging the response of organs, tissues and neoplastic growth to therapy, in the life science community.

### SUMMARY OF THE INVENTION

In one embodiment, the invention provides a hyperspectral imaging system comprising: an electromagnetic energy source; coupled to a prism and reflector imaging spectroscopy system (PARISS) equipped with an imaging probe comprising a plurality of customized bundle of structured optical fibers that can be uses as a spatially resolved imaging probe.

In another embodiment, provided herein is a method of acquiring in-vivo hyperspectral image from a subject, comprising: selecting a field of view (FOV) of the subject; attaching a plurality of customized, bundle of structured optical fibers that can be uses as a spatially resolved imaging probe, wherein the customized, spatially structured fiber-optic probes are operably linked to a hyperspectral imaging system comprising: an electromagnetic energy source; coupled to a prism and reflector imaging spectroscopy system (PARISS); illuminating the field of view using the hyperspectral imaging system; and collecting an in-vivo hyperspectral image.

In one embodiment, the invention provides method of monitoring neoplasia of a tissue in a subject, comprising the step of obtaining a hyperspectral image of a field of view of an area sought to be monitored; and comparing the image to a standard.

In another embodiment, the invention provides a method of imaging a natural history or response to therapy of lesions of the skin, oropharynx, esophagus, bladder, or intra-abdominal lesions accessed through laparoscopy, comprising the step of obtaining a hyperspectral image of a field of view of an area sought to be monitored in the lesions of the skin, oropharynx, esophagus, bladder, or intra-abdominal lesions accessed through laparoscopy; and comparing the image to a standard.

In another embodiment, the invention provides a library of spectral signatures of field of view obtained from the methods described herein.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1 shows a photograph of the prototype in vivo MACRO-PARISS hyperspectral imaging system. Arrows indicate the fiber-optic imaging probe, an imaging spectrometer, a QICAM camera, an observed image camera, and laptop that controls the cameras (High intensity tungsten halogen lamp not shown);

FIG. 2 wherein the solid line shows the MIDL wavelength calibration lamp spectrum after normalization, and the dotted line before normalization. Note that following normalization, the spectral range around 436 nm and 900 nm increased in intensity, and the 546 nm line was almost unchanged;

FIG. 3 wherein the line with red diamond markers shows the QE profile of the camera. The line with blue diamonds presents the profile as rendered by the MACRO-PARISS system and is a convolution of the QE of the camera, the actual spectral profile of the lamp, coatings, fiber optic, and all other optical elements. The curve with open circles is the profile in the NIST-certified profile; and the superimposed curve with solid circles is the normalized MACRO-PARISS profile of the lamp following normalization;

FIG. 4 shows a photograph of the 9 areas designated for hyperspectral imaging. Note the clear juxtaposition of vascular and non-vascular areas. This consistent vascular anatomy to allowed for analysis of inter- and intra-animal imaging variability of generated spectral data. Acquiring images of the same area on different mice as well as imaging the same area on the same mouse but on different days was possible;

FIG. 5 shows (A) radiometric Master Spectral Library (MSL). Light colors tend towards areas in the FOV without vasculature and those in redder colors indicate the presence of blood vessels. (B) Using a MCC of 99%, spectral histograms depicting percent composition of spectral objects were generated from Area 1 (vascular). The components of the histogram are a result of our ability to display and characterize spatial heterogeneity. Images were acquired on three separate days on the same mouse. The spectral image to the far right represents the same spectral data in spectral format. Note the consistency in spectral signatures in histogram and spectral format, supporting the notion that this HSI system can generate reproducible signatures over comparable regions of interest. Spectral histograms for all nine areas were similarly generated for analysis;

FIG. 6 shows spectral histograms of Area 1 (top row-vascular) and Area 4 (bottom row-non-vascular). Note the spectral differences in vascular regions of the skin when compared to non-vascular regions. All spectral histograms were generated from an MSL at 99% MCC;

FIG. 7 shows spectral histograms of Areas 5 and 9 closely resembled a vascular signature. Note the “hybrid” spectral histogram displayed in Areas 6 and 7. Spectral images are displayed next to their respective histogram. Movement of the probe from a vascular to a non-vascular area results in a significant and step-wise change in spectral signatures;

FIG. 8 shows reversion of a vascular spectral signature (Area 1) to a non-vascular spectral signature after the removal of its blood supply. Area 4 did not change before and after the removal of the blood supply. All spectral histograms are composites of triplicate imaging acquisitions;

FIG. 9 shows an embodiment of spatial distribution of the PARISS remote fiber optic probe. Different fibers collect light from different areas of the sample, fibers that are mapped to the slit can then be reconstructed to produce an image. Light source (1), Lens focuses light source on illumination fibers (2), End of fiber tip showing illumination fibers. Can be arrayed as a bundle or a line depending on illumination source (3), Bifurcated fiber integrates illumination and light collection fibers (4) End tip of fiber that touches tissue (proximal end) (10). Shows illumination fibers and collection fibers (5). Tip of collection fibers (distal end) arrayed along a “line” (6) Lens to transfer image of collection fibers onto the entrance slit (8) of the PARISS spectrometer (9) (7). Entrance slit of PARISS spectrometer (9) (8). PARISS spectrometer (9); and

FIG. 10 shows spectral analysis of an area of “Blood at 45 degrees tilt”, MCC=99.5%. A. Proximal fiber tip, that touches tissue, identifying the location of individual fibers arrayed at the distal end and focused onto the slit of the spectrometer. B. Array of fibers originating at the proximal end of the fiber bundle. Each numbered fiber at the distal end can be traced to a given location at the proximal end. C. Color codes corresponding to “fingerprint” spectra in the MSL. D. A histogram showing the ratio of MSL spectra that meet a given minimum correlation coefficient (MCC). In this case the histogram is that of the acquisition shown in (E). E. A spectral image taken through the PARISS spectrometer of the distal end of the fiber bundle. Software assigns a color code according to whether a given MMC. No color code is assigned if the spectrum from a given fiber fails to meet a MCC target.

### DETAILED DESCRIPTION OF THE INVENTION

This invention relates in one embodiment to the use of in-vivo hyperspectral imaging to monitor angiogenesis. In another embodiment, the invention relates systems and methods of obtaining hyperspectral images of a field of view comprising an area sought to be monitored.

In one embodiment, Hyperspectral imaging (HSI) refers to a form of optical imaging that is used by the Remote Earth Sensing Community for environmental studies, and is typically used in radiometric mode. In another embodiment, HSI refers to any instrument that faithfully digitizes an analog spectrum presented by the field of view (FOV), requiring in another embodiment, that each spectrum be characterized by a large number of data points. In one embodiment, in the case of a spectrometer, a large number of data points refers to greater than 600 wavelength data points over the entire spectral range. This correlates with a spectral resolution that will be better than 5 nm at wavelengths below 600 nm nad better than 10 nm at wavelengths up to 920 nm.

In one embodiment, the induction of new blood vessel formation is a prominent feature of solid tumors and it is well established that tumor size beyond 2 mm³ requires the construction of a vessel network. Mean vascular density is correlated in another embodiment, to progression of cutaneous melanoma. In one embodiment, a correlation between overall survival in melanoma patients and tumor microvessel density exists, supporting the notion of a vascular density gradient and vertical tumor progression.

In another embodiment, a HSI system can generate quantifiable spectral differences in a reproducible manner between vascular and non-vascular regions of skin. Accordingly and in one embodiment, provided herein is a hyperspectral imaging system comprising: an electromagnetic energy source; coupled to a prism and reflector imaging spectroscopy system (PARISS) equipped with an imaging probe comprising a plurality of customized bundle of optical fibers that can be uses as probes. In one embodiment, diffraction gratings diffract light into second and higher orders with the consequence that efficiency degrades and longer wavelengths can be polluted by commingled second order diffraction, conversely, Prisms refract light into one order and present the highest possible light transmission. In another embodiment, the PARISS system used in the methods provided herein, is prism based and presents ALL wavelengths simultaneously, therefore, in one embodiment movement in the tissue imaged does not affect a spectral characterization.

In one embodiment, the HSI system used in the methods and compiling of the spectral images and libraries described hereinbelow incorporates a high intensity tungsten halogen lamp coupled with a PARISS (Prism and Reflector Imaging Spectroscopy System)—and customized bundle of structured optical fibers that can be uses as a spatially resolved imaging probe. The high intensity tungsten lamp is used in another embodiment, as the source of light because since it provides a greater amount of red intensity, which in one embodiment, is ideal for penetrating and probing living tissues. In another embodiment, since many variables within an in vivo skin section such as pH, protein interactions, temperature, and ionic concentration preclude a linear mixture of spectral colors, the PARISS system used in the methods and compiling of the spectral images and libraries described hereinbelow is able to process non-linear high resolution spectral data, thereby making it suitable for the evaluation sought.

In one embodiment, the light source is composed of a simple light bulb, or a flash lamp, or another light source or combination of sources, or it may be a complex form including gateable or triggerable electronics, a light source, a filter element, a transmission element such as an optical fiber, a guidance element such as a reflective prism, and other elements intended to enhance the optical coupling of the light from the emitter to the skin or FOV under study in other embodiments. The light source may be continuous, pulsed, or even analyzed as time, frequency, or spatially resolved. The emitter may consist of a single or multiple light emitting elements. In another embodiment, optical coupling used to operably link the light source, or the imaging probe refers to the arrangement of a light source (or light detector) in such a way that light from the source (or detector) is transmitted to (or detected from) the FOV, allowing passage through the tissue and possible interaction with a contrast agent or in another embodiment, a detectable molecular probe or marker. This may require in one embodiment, the use of optical elements such as lenses, filters, fused fiber expanders, collimators, concentrators, collectors, optical fibers, prisms, mirrors, or mirrored surfaces and their combination.

As would be appreciated by a person skilled in the art, the use of LED light source of various colors is also encompassed by the methods and systems described herein, to produce in certain embodiments, off-white light with emphasis on certain ranges of the spectrum, thereby obtaining a different response spectra. In certain embodiment, the light source used in the methods and compositions described herein, is optimized to the tissue being imaged, or the pathology observed or monitored. It will be appreciated that the spectra used to image the response of a angiogenesis on the skin, may be different in one embodiment from the optimal spectra used to obtain an intra-abdominal lesions accessed through laparoscopy.

In one embodiment, spectral images are generated using a charge-coupled device (CCD) detector. In another embodiment, the plurality of customized bundle of structured optical fibers that can be uses as a spatially resolved imaging probes used in the systems provided herein are spatially discrete. In another embodiment, the prism and reflector imaging spectroscopy system comprises an imaging spectrometer integrated with a camera; and a second camera, which in certain embodiments is QICAM camera, acting as an observed image camera. In one embodiment, the plurality of customized bundle of structured optical fibers that can be uses as a spatially resolved imaging probes comprise illumination probes and signal collection probes, wherein in yet another embodiment the collection probes to are arrayed along a slit thereby collecting images onto an entrance slit in the a prism and reflector imaging spectroscopy system

In one embodiment, establishing use of the systems provided herein in discriminating vascular and non-vascular areas allows for the assessment of physiologic and pathologic processes in the tumor microenvironment. Utilizing the systems provided herein coupled to a bundle of structured optical fibers that can be uses as a spatially resolved imaging probe, the reproducibility and robustness of the spectral signatures derived from comparable regions of interest is evident.

In one embodiment, performance optimization of the HSI system in generating reproducible and unique spectral signatures is determined by three factors. In one embodiment a high intensity tungsten lamp, used as a light source, allows the emission of light in the red range, which experiences less absorption by tissue. In another embodiment, an appropriate acquisition parameters are set, which increase the signal-to-noise ratio thereby reducing single acquisition variability. In one embodiment, when creating the master spectral library (MSL), only spectra that comes from the center of the signal collection fibers is entered, leading to a more robust MSL.

In one embodiment, the term “Signal to Noise ratio” refers to the ratio of the strength of a target signal to the background noise. This can be increased either by improving the target signal in one embodiment, or by reducing the background noise or their combination in other embodiments.

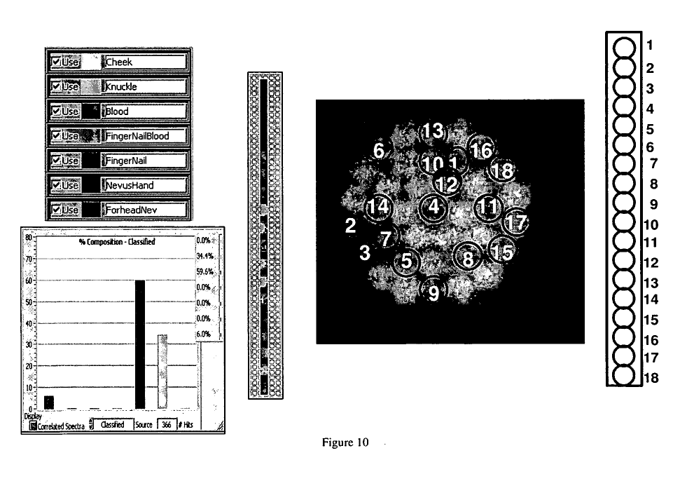

In one embodiment, the systems described herein, used in the methods and compiling of the spectral images and libraries described herein comprise a plurality of fiber optic probes, consisting in one embodiment of a plurality of illumination fibers and signal collecting fibers. In another embodiment, the fiber-optic probe consists of 17 illumination fibers and 18 signal collection fibers, which in one embodiment, are randomly distributed, with each fiber having in another embodiment, a 50-micron core. Signal collection fibers are arrayed in one embodiment, along a slit and imaged onto the PARISS entrance slit. In one embodiment, each fiber delivers spectral information from a different point in the FOV. In another embodiment, the in vivo delivery of spatially discrete information differentiates the system described herein from traditional spectral modalities that acquire a single, homogenized, all encompassing, data-point. In one embodiment, the number of illumination fibers and signal collection fibers are optimized based on the area of the FOV sought to be imaged, the core dimensions and the resolution sought. In one embodiment, the fiber-optic probe consists of 15 illumination fibers and 16 signal collection fibers. In another embodiment, the fiber-optic probe consists of 20 illumination fibers and 21 signal collection fibers. In another embodiment, the fiber-optic probe consists of no less than 15 illumination fibers and no less than 15 signal collection fibers. (See e.g., FIG. 9)

In one embodiment, spatially structured fiber-optic probes consisting of illumination fibers and signal collection fibers, which in one embodiment, are randomly distributed, with each fiber having in another embodiment, a core that is optimized for the underlying application, the size of the FOV the location and other factors in other discrete embodiment. As would be appreciated by a person skilled in the art, the core diameter can be adjusted to provide the optimal signal to noise ratio.

In one embodiment, the fiber-optic probe consists of 15 illumination fibers and 16 signal collection fibers may also be partially attached to a cuff as well. The 15 illumination fibers are cuffed together in one embodiment and placed on a tissue or organ of the subject in another embodiment of the methods and systems described herein. Likewise and in another embodiment, the 16 signal collection fibers may also be partially attached to a cuff placed on the same or different body organ or tissue, such as skin in another embodiment. While the cuff encircles the entire number of fibers, it is within the scope of the invention (and definition of the term ‘cuff’) to include a series of discrete cuffs or patch-like elements distributed around the tissue or organ of the subject, as points to which the illumination or collection fibers are attached.

In one embodiment, the embodiments of the systems described hereinabove and their inherent variables are used to carry out the methods provided herein. Accordingly and in one embodiment, provided herein is a method of acquiring in-vivo hyperspectral image from a subject, comprising: selecting a field of view (FOV) of the subject; attaching a plurality of customized remote fiber-optic probes, wherein the customized remote fiber-optic probes are operably linked to a hyperspectral imaging system comprising: an electromagnetic energy source; coupled to a prism and reflector imaging spectroscopy system (PARISS) equipped with an imaging probe; illuminating the field of view using the hyperspectral imaging system; and collecting an in-vivo hyperspectral image.

In another embodiment, provided herein is a method of acquiring in-vivo hyperspectral image from a subject, comprising: selecting a field of view (FOV) of the subject; attaching a plurality of customized remote fiber-optic probes, wherein the customized remote fiber-optic probes are operably linked to a hyperspectral imaging system comprising: an electromagnetic energy source; coupled to a prism and reflector imaging spectroscopy system (PARISS) equipped with an imaging probe; illuminating the field of view using the hyperspectral imaging system; collecting an in-vivo hyperspectral image; and compiling a unique spectral signature of the field of view.

In one embodiment, each fiber in the bundle of the systems probe presents light to the PARISS slit according to its respective location in the FOV, providing in another embodiment spatial information and allows to characterize field heterogeneity and compile a spectral signature of the FOV. In one embodiment, the spectral signature comprises the location and amplitude of signal from every point in the FOV of the probe, thereby generating a hyperspectral data cube consisting in one embodiment of wavelength, or spatial and graphic information and their combination in another embodiment (See e.g. FIG. 10).

In one embodiment, the spatial resolution of the fibers is used for low resolution imaging. In another embodiment, the systems and methods provided herein have a considerable advantage over single, or pairs, of fibers.

In one embodiment, HSI systems used in the methods and compiling of the spectral images and libraries described hereinbelow comprising the fiber-optic attachment described hereinabove in the form of a plurality of customized bundle of structured optical fibers that can be uses as a spatially resolved imaging probes comprise illumination probes and signal collection probes, provides reproducible vascular or non-vascular signatures and their combination in other embodiments. In one embodiment, as the FOV moves from vascular to non-vascular areas, the acquired spectra change in a step-wise predictable fashion, allowing the use of the methods and compiling of the spectral images and libraries described herein.

In one embodiment, the invention provides images obtained by the embodiments of the methods described herein. In one embodiment, provided herein is a method of monitoring angiogenesis in a subject in response to a therapeutic treatment, comprising obtaining a hyperspectral image of a field of view of the area sought to be monitored; and monitoring changes in the spectral image in response to the therapeutic treatment. In one embodiment, obtaining a hyperspectral image of a field of view of the area sought to be monitored comprises attaching a plurality of customized remote fiber-optic probes to the area sought to be monitored, wherein the customized remote fiber-optic probes are operably linked to a hyperspectral imaging system comprising: an electromagnetic energy source; coupled to a prism and reflector imaging spectroscopy system (PARISS) equipped with an imaging probe; illuminating the field of view using the hyperspectral imaging system; and collecting an in-vivo hyperspectral image. In one embodiment, the PARISS data processing algorithms is used for thresholding in association with histograms. These can be set in another embodiment, to “grade” the level of angiogenesis by providing ratios of spectrally vascular to spectrally non-vascular regions.

In another embodiment, provided herein is a method of monitoring angiogenesis in a subject in response to a therapeutic treatment, comprising obtaining a hyperspectral image of a field of view of the area sought to be monitored; and monitoring changes in the spectral image in response to the therapeutic treatment, whereby obtaining a hyperspectral image of a field of view of the area sought to be monitored comprises attaching a plurality of customized remote fiber-optic probes to the area sought to be monitored, wherein the customized remote fiber-optic probes are operably linked to a hyperspectral imaging system comprising: an electromagnetic energy source; coupled to a prism and reflector imaging spectroscopy system (PARISS) equipped with an imaging probe; illuminating the field of view using the hyperspectral imaging system; collecting an in-vivo hyperspectral image; and compiling a unique spectral signature of the field of view.

In one embodiment, the gradual spectral signature change, which occurs in a predictable fashion based on the relevant regional presence of vascularity, the HSI system described herein provides a capability to monitor subtle changes in tumor vascularity before and after therapeutic intervention. In one embodiment, the angiogenesis sought to be monitored is associated with cutaneous inflammatory and cancerous lesions. In another embodiment, the angiogenesis sought to be monitored is associated with gastrointestinal lesions via colonoscopy or esophagoscopy, and during surgery such as in lymph node assessment. In another embodiment, the angiogenesis sought to be monitored is associated with differentiating oxy- and deoxyhemoglobin as a surrogate marker of tumor hypoxia.

In certain embodiments, the use of spatially resolved fibers or spatially structured fiber probes in other embodiments, or imaging probe as described herein, enables the characterization of tissue in which it is expected that at a micro level some areas will be vascular and some not. The ratio of vascular to non vascular areas, as resolved using the methods described herein, at a micro level, assists in another embodiment, in determining the spatial borders of a tumor, which in another embodiment, may be used in automated cancer detection.

In one embodiment, provided herein is a method of monitoring angiogenesis in a subject in response to a therapeutic treatment, comprising obtaining a hyperspectral image of a field of view of the area sought to be monitored; and monitoring changes in the spectral image in response to the therapeutic treatment, wherein obtaining a hyperspectral image of a field of view of the area sought to be monitored comprises attaching a plurality of customized remote fiber-optic probes to the area sought to be monitored, wherein the customized remote fiber-optic probes are operably linked to a hyperspectral imaging system comprising: an electromagnetic energy source; coupled to a prism and reflector imaging spectroscopy system (PARISS) equipped with an imaging probe; illuminating the field of view using the hyperspectral imaging system; collecting an in-vivo hyperspectral image; compiling a unique spectral signature of the field of view; and comparing the spectral signature of the field of view with the spectral signature of the same field of view obtained from a subject exhibiting angiogenesis in one embodiment, or a subject not exhibiting angiogenesis in another embodiment.

In one embodiment the methods of data acquisition provided herein, may be useful to the clinic as a reliable adjunct to the pathologist, oncologist, surgeon and dermatologist in monitoring tumor response after therapeutic intervention.

In one embodiment, each fiber in the bundle or cuff in another embodiment, presents light to the PARISS slit according to its location in the FOV. Spectral and illumination variations from fiber to fiber are dependant on; localized variations in the FOV (spatial inhomogeneity) in one embodiment, or variations in tilt, variations in pressure, individually damaged fibers, motion or their combination in other embodiments. Accordingly, in certain embodiments the spectral image obtained using the systems described herein in the methods provided herein is adjusted or normalized to the factors described herein, to be compared to a standard spectral image library. In one embodiment once images obtained using the systems described herein in the methods provided herein are normalized, they are incorporated into the libraries provided herein.

When the target tissue is dry in one embodiment, an index matching fluid is used such as oil in one embodiment, or glycerin in another, to better couple the proximal end of the fibers with the tissue under examination. In another embodiment this increases light transmission and reduce the effects of wear on the ends of the fibers”. In another embodiment, an index matching material is disposed between the dry tissue and the proximal end of the fibers, for maintaining a constant and matched index for the light directed into the tissue and the light reflected from the tissue. In one embodiment, an index matching gel reduces large index of refraction changes that would occur normally between a dry tissue and a gap of air. These large changes result in Fresnel losses that are especially significant in a reflectance based analysis, which creates significant changes in the spectral signal. According to one embodiment of the present invention, the indexing matching material is a chloro-fluoro-carbon gel. This type of indexing material has several favorable properties. First, the chloro-fluoro-carbon gel minimally impacts the spectral signal directed through the gel. Second, this indexing matching material has a high fluid temperature point so that it remains in a gel-like state during the analysis and under test conditions. Third, this gel exhibits hydrophobic properties so that it seals the sweat glands so that sweat does not fog-up (i.e., form a liquid vapor on) proximal end of the fiber (tip). And fourth, this type of index matching material will not be absorbed into the stratum corium of skin during the analysis.

In one embodiment, provided herein is a library of spectral signatures of field of view obtained from a subject undergoing angiogenesis therapy, wherein the field of view comprises a tumor area, a vascular area, a non-vascular area, a cutaneous inflammatory lesion, a cancerous lesion, a gastrointestinal lesion or a combination thereof.

In one embodiment, the method described herein, are capable of being applied to other clinical applications such as early detection of neoplasia assessing remission or recurrence, or in other embodiment, monitoring either or both the natural history or response to therapy of lesions of the skin in one embodiment, or oropharynx, esophagus, bladder or potentially intra-abdominal lesions accessed through laparoscopy in other discrete embodiments. In another embodiment, the methods of obtaining a hyperspectral images using the systems described herein, can collect spectral signatures that are attributed to angiogenesis in one embodiment, or other alterations in a field of view, including metabolic changes, necrosis, inflammation, or neoplastic transformation in other discrete embodiments of the methods described herein. The methods and systems described herein, are used in another embodiment to provide evidence of therapeutic response to cancer therapy (chemotherapy or various forms of radiation) or other therapies such as photodynamic therapy in certain embodiments, reflected as changes in spectral characteristics due to altered angiogenesis or other metabolic changes or necrosis as described.

In one embodiment, provided herein is a method of monitoring neoplasia of a tissue in a subject, comprising the step of obtaining a hyperspectral image of a field of view of an area sought to be monitored; and comparing the image to a standard, whereby the step of obtaining a hyperspectral image of a field of view of the area sought to be monitored comprises attaching a plurality of customized remote fiber-optic probes to the area sought to be monitored, wherein the customized remote fiber-optic probes are operably linked to a hyperspectral imaging system comprising: an electromagnetic energy source; coupled to a prism and reflector imaging spectroscopy system (PARISS) equipped with an imaging probe; illuminating the field of view using the hyperspectral imaging system; and collecting an in-vivo hyperspectral image.

In another embodiment, provided herein is a method of imaging a natural history or response to therapy of lesions of the skin, oropharynx, esophagus, bladder, or intra-abdominal lesions accessed through laparoscopy, comprising the step of obtaining a hyperspectral image of a field of view of an area sought to be monitored in the lesions of the skin, oropharynx, esophagus, bladder, or intra-abdominal lesions accessed through laparoscopy; and comparing the image to a standard, whereby the step of obtaining a hyperspectral image of a field of view of the area sought to be monitored comprises attaching a plurality of customized remote fiber-optic probes to the area sought to be monitored, wherein the customized remote fiber-optic probes are operably linked to a hyperspectral imaging system comprising: an electromagnetic energy source; coupled to a prism and reflector imaging spectroscopy system (PARISS) equipped with bundle of structured optical fibers that can be uses as a spatially resolved imaging probe; illuminating the field of view using the hyperspectral imaging system; and collecting an in-vivo hyperspectral image.

In one embodiment, the compiled unique hyperspectral signature images are used to make a master spectral library. The term “library of hyperspectral images” refers in one embodiment to the collection of hyperspectral data that is being generated employing the systems and methods disclosed herein. The basis of choice of the library of hyperspectral images used in the methods described herein, will vary in another embodiment, with the application.

In one embodiment, the library provided herein is used for identifying the type of neoplastic process, or in other embodiments the degree, responsiveness to treatment, pathology, and the like in other embodiments. The fact that complete spectra are available, allows in one embodiment, to identify the different responses. The samples upon which the library is built may be categorized, or otherwise diagnosed, as diseased or non-diseased by a variety of methods. In one embodiment, a pathologist utilizes conventional procedures to make such a determination. In another embodiment, the diagnosis is made by conventional histological techniques, including conventional histochemical and/or biochemical techniques. In certain embodiment, following such diagnosis, the spectral image of the sample is obtained using the systems and methods described herein. Accordingly, and in one embodiment, the database, or library, may include a digital spectrum library, and/or a library of desired spectral features as described herein, stored in a computer. In other forms of the invention, the spectroscopic data may be compared graphically or by other similar methods. If desired, a background adjustment may be made by having the software subtract from the spectra analyzed the background reflectance spectra of normal tissue, both with and without stain, including a baseline spectrum of the patient's normal tissue.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

### Hyperspectral Imaging System Instrumentation

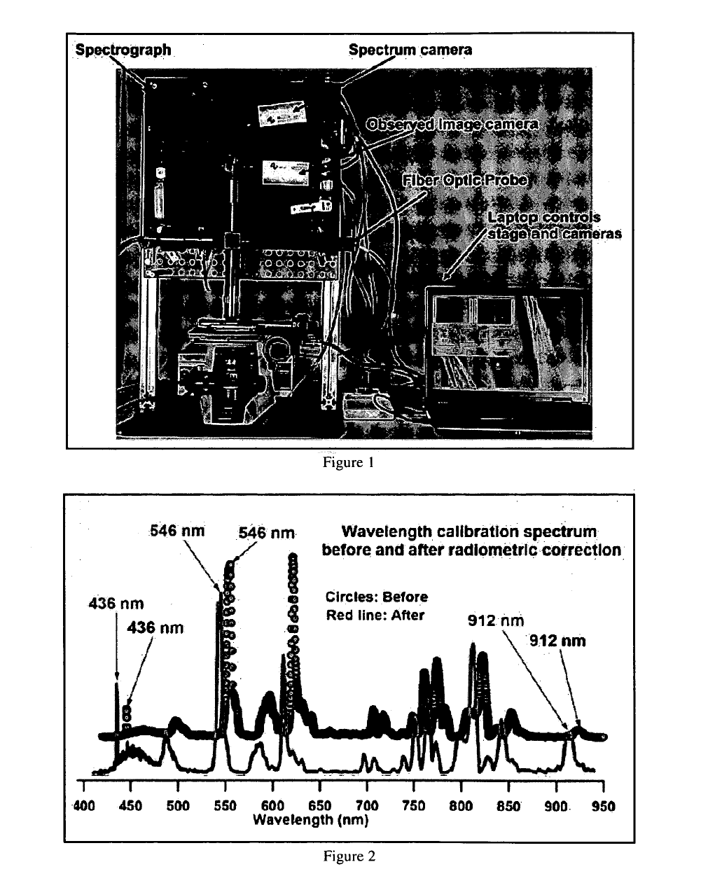

The in vivo hyperspectral imaging system consists of a spatially discrete, multi-fiber optic imaging probe (LightForm, Inc., Hillsborough, N.J.), an imaging spectrometer integrated with a QICAM camera (Q Imaging, Burnaby Canada), and a second QICAM acting as an observed image camera (FIG. 1).

The fiber-optic probe consists of 17 illumination fibers and 18 signal collection fibers, randomly distributed, with each fiber having a 50-micron core. Signal collection fibers were arrayed along a slit and imaged onto the PARISS entrance slit. Each fiber delivered spectral information from a different point in the FOV.

The MACRO-PARISS system (LightForm, Inc., Hillsborough, N.J.) is a prism based imaging spectrometer that originated within the remote Earth sensing community. This system was chosen because of its very high light transmission (>90%) characteristics typical of prism systems. The imaging spectrometer portion operates in spectrograph configuration in which all wavelengths between 365 and 920 nm are presented simultaneously. Acquiring all wavelengths within a single fast acquisition accommodates movement in the FOV without affecting the integrity of a spectral acquisition. In this study, a range of 450 to 920 nm was chosen.

### Calibration, Normalization and Validation

The MACRO-PARISS spectrometer was first wavelength calibrated using a multi-ion discharge lamp (MIDL), (LightForm, Inc., Hillsborough, N.J.) that emits Hg+, Ar+ and inorganic fluorophores (FIG. 2). Each pixel in a column of the spectrum CCD corresponds to a specific wavelength. The MIDL lamp provides the absolute wavelength information provided by the ion emission lines. The MACRO-PARISS software provides an algorithm that matches the spectral features to pixel with subsequent calibration of the entire spectrometer. The wavelength accuracy was validated to be better than 0.5 nm over the entire spectral range.

Spectral resolution was confirmed by measuring the number of pixels that covered the full width at half maximum (FWHM) at the 436 nm Hg line. The wavelength spread at FWHM was determined to be 1.2 nm+/−0.25 nm.

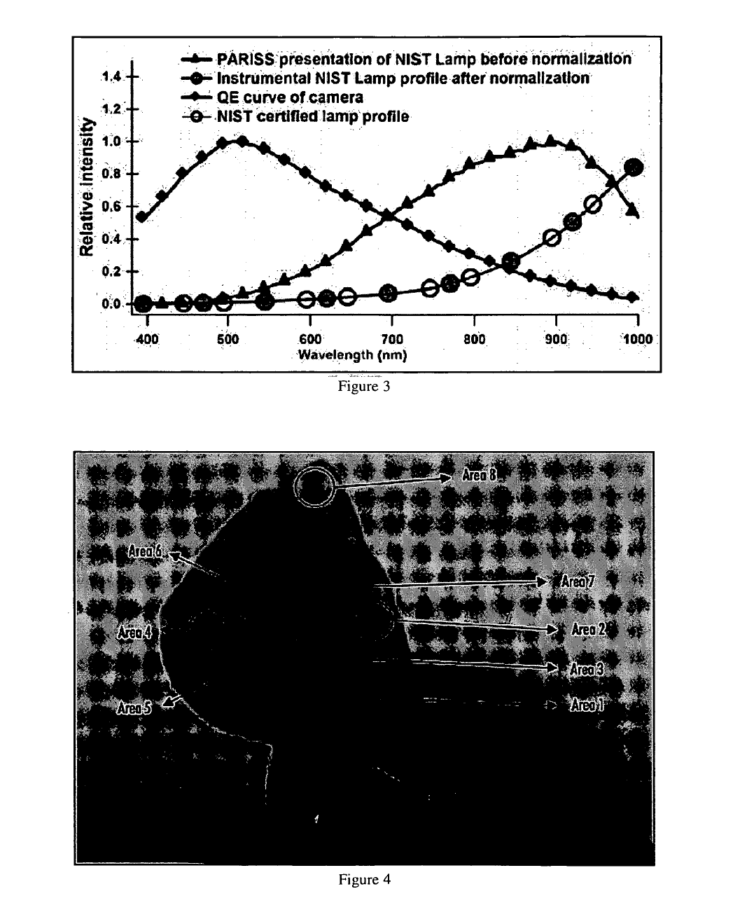

The MACRO-PARISS spectrometer was then normalized to remove instrumental contributions due to the Quantum Efficiency (QE) of the camera, coatings on lenses, the fiber-optic and prism transmission properties, and reflectivity of mirrors in the system. A NIST-certified halogen lamp (NCHL) (Model LS-1-Cal, Cert #1013, Ocean Optics, Inc, Dunedin Fla.) was used that was supplied with a spectral profile in ASCII listing wavelength versus power (μW/cm²/nm). This is the profile any radiometric spectrometer would report when characterizing this lamp. The MACRO-PARISS software incorporates the algorithm to enable this normalization. FIGS. 2 and 3 show the profile of the NCHL reported by the MACRO-PARISS spectrometer before and after correction, and the QE of the camera. The corrected profile is seen to be a perfect match to the profile shown on the lamp's certificate.

### Light Source, Calibration and Methods

A high intensity tungsten halogen lamp set to the highest brightness was used as the light source for the fiber-optic probe to increase signal-to-noise data at the ends of the spectral range and for greater red intensity. The wavelength range from 450 to 920 nm, especially in the near IR, above 650 nm, experiences less absorption in living tissues and would theoretically provide valuable tissue spectral information otherwise not accounted for with other light sources.

The entire spectral system, including the probe, was wavelength-calibrated daily prior to imaging experiments to ensure accuracy using the MIDL emission source as described above. Spectral sensitivity was checked in the advanced display mode with amplitude plotted against wavelength at the time of calibration. After wavelength calibration, the system was normalized radiometrically through the fiber-optic probe with the NIST certified, halogen light source. A correction curve was then generated in the MACRO-PARISS software to correct all acquisitions to the standard.

Since each fiber in the bundle presents light to the MACRO-PARISS slit according to its respective location in the FOV, an attempt was made to account for a heterogeneous FOV by using a fiber-optic probe with a relatively higher number of individual fibers—as described in the instrumentation section. This would provide spatial information and allow the characterization of field heterogeneity. The fact that spatial information is delivered in vivo differentiates this system from traditional spectral modalities that acquire a single, homogenized, all encompassing, data-point.

Additionally, the ability to map the 18 collection fibers to the slit in order to generate a low resolution graphic image is recognized and being addressed. Determining the location and amplitude of signal from every point in the FOV of the probe, becomes possible, generating a hyperspectral data cube consisting of wavelength, spatial and graphic information.

### In Vivo Spectral Acquisition and Acquisition Parameters

The branching artery of the SCID mouse ear was used as the model because it has adjacent regions of vascular and non-vascular skin. Nude mice were selected due to their ears having a consistent vascular anatomy, allowing for a clear comparative analysis between vascular and non-vascular acquisitions. Additionally, it was anticipated that hair would contribute to a spectral signature and hence, nude mice would circumvent this issue for these examples.

Using an Institutional Animal Care and Use Committee (IACUC)-approved protocol, three nude mice were anesthetized with a ketamine/xylazine solution (125 mg/kg and 15 mg/kg, respectively) via intraperitoneal injection. Subsequently, the probe was gently placed with uniform pressure on the skin, ninety degrees to the selected region for spectral acquisition.

### Parameters to Reduce Single Acquisition Variability

Specific acquisition parameters were determined based on an inverse relationship between movement variability of the user and acquisition variability. The fact that all wavelengths were acquired simultaneously ensured that there was no variability from wavelength to wavelength; however, movement of the probe due to the user could result in differences in the illuminated area. The greater the acquisition times the lesser the acquisition variability but the greater the chance of movement by the user.

An attempt was made to address the problem of spectral sampling variability by using standard analytical sampling procedures. Each in vivo skin acquisition consisted of 5 co-added 25 millisecond acquisitions 5-times; resulting in an improvement in the signal-to-noise ratio (S/N) of a factor of 2.2 (square root of 5). Each area was sampled 5 times, which was found to be an optimal balance where any investigator could hold still, without compromising the acquisition quality and hence, maintain a well-defined S/N ratio.

A total of nine distinct skin regions (Areas 1-9) were imaged in triplicate on different days and subsequently compared (FIG. 4). At the time of sacrifice, vascular and non-vascular skin areas were imaged before and after stripping the vascular blood supply to the ear via a 1 cm surgical incision made at the base of the ear, with subsequent drainage of the blood to gravity.

### Image Analysis Software

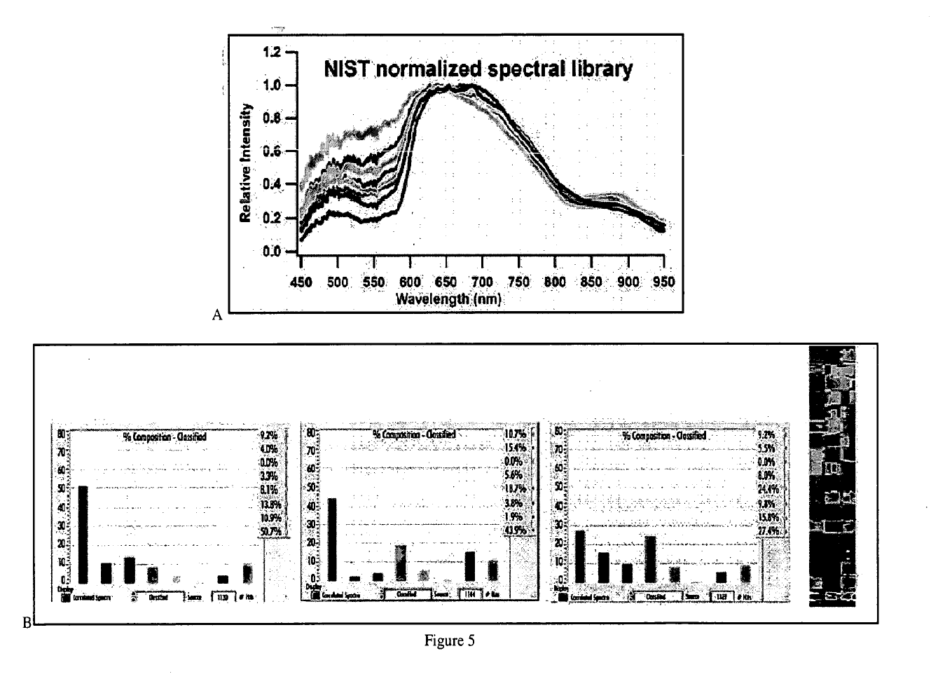

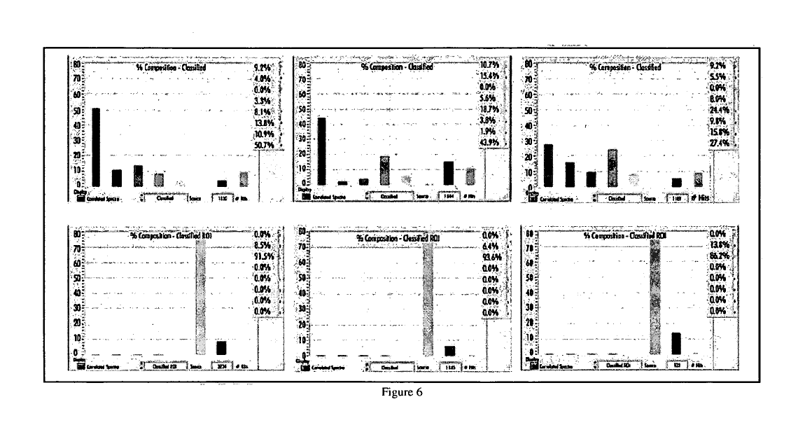

It was assumed that many physiologic variables within an in vivo skin region would preclude a linear mixture of spectral signatures. The MACRO-PARISS system accounts for this physiological challenge since it is able to process non-linear spectral data from spectral objects localized near each other. Master spectral libraries (MSL) were generated from acquired spectral data through a supervised classification using a spectral waveform cross correlation analysis (SWCCA) algorithm. All library spectra were saved in radiometric format (FIG. 5A). Spectra corresponding to more vascular areas were pseudo-colored in redder colors and non-vascular areas in bluer colors. Note that a minimum was formed around 550 nm that probably corresponds to absorption by red blood cells.

While previous studies have used a combination of automated and supervised classification schema for generating MSL, a more conservative strategy was taken, since an automated classification would have included spectral data from the peripheral regions of the fiber-optic collection fibers, leading to less robust spectral signatures. Each spectrum that was manually added to the MSL was designated a pseudo color. All spectra from the original FOV were then correlated with the MSL, set to a minimum correlation coefficient (MCC) of 99%. Only when the spectrum from the FOV correlated at 99% or greater with an MSL spectrum, did the pseudo-color replace the gray scale pixel. Additionally, histograms of MSL pseudo-color members were generated to present spectral ratios within the FOV and were used to compare vascular and non-vascular skin regions.

### Example 1

### Reproducibility and Robustness of Acquired MACRO Hyper-Spectral Signatures

The approach to testing the reproducibility of the MACRO-HSI system consisted of imaging the vascular anatomy of the ear, since it allowed to image the same designated areas on the same mouse on different days, as well between mice. This approach would allow to assess intra- and inter-animal imaging variability. All 4 areas of the ear were imaged in triplicate on different days.

The Master Spectral Library (MSL) was used to generate all ear spectral histograms. All spectra from the original FOV were correlated with the MSL, set to a minimum correlation coefficient (MCC) of 99%. Spectra from the spectral image from light captured exclusively from the center (core) of the 18 collection fibers were manually added. This served to create a more robust spectral library since it did not include spectral data from the walls of the collection fibers. Each fiber delivered a spectral signature from a different point on the FOV.

FIG. 5B clearly demonstrates the consistency in spectral histograms generated from the nine areas of variable vascularity within the mouse ear.

### Example 2

### In Vivo Hyperspectral Differentiation of Vascular and Non-Vascular Regions of the SCID Mouse Ear

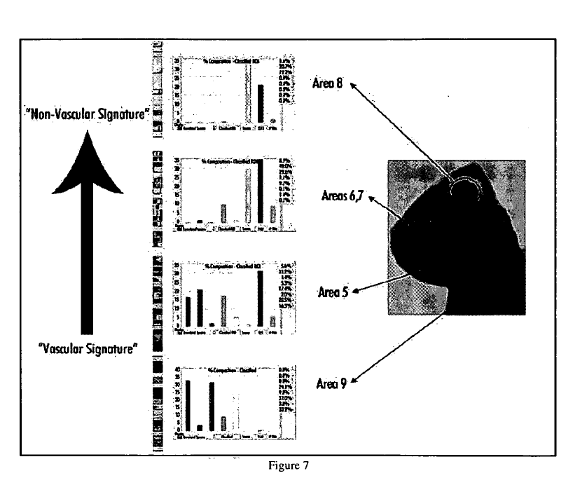

Spectral histograms of vascular and non-vascular regions of the skin were compared. FIG. 6 illustrates the spectral differences in vascular regions (Areas 1 and 3) of the skin as compared to non-vascular regions (Areas 2 and 4). The results indicate that similar regions of interest with respect to vascularity consistently generated a unique spectral signature. Further characterization of the uniqueness of the vascular spectral signature was made by imaging additional regions of the ear, Areas 5-9. These additional areas were chosen for analysis because they all included visible vascularity but differed with respect to the level of gravity pull and their distance from the base vascular supply. It was sought to image a gradation of vascular prominence as a potential surrogate marker for vessel formation and retraction.

With these additional areas, it was hypothesized that Areas 5 and 9 would generate spectral signatures similar to 1 and 3 (i.e., a vascular signature) while Area 8 would closely resemble Areas 2 and 4 (i.e., a non-vascular signature). Areas 6 and 7 might behave in one of two ways: either 1) these areas would capture a signature that would be a combination of vascular and nonvascular signatures or 2) capture a vascular signature.

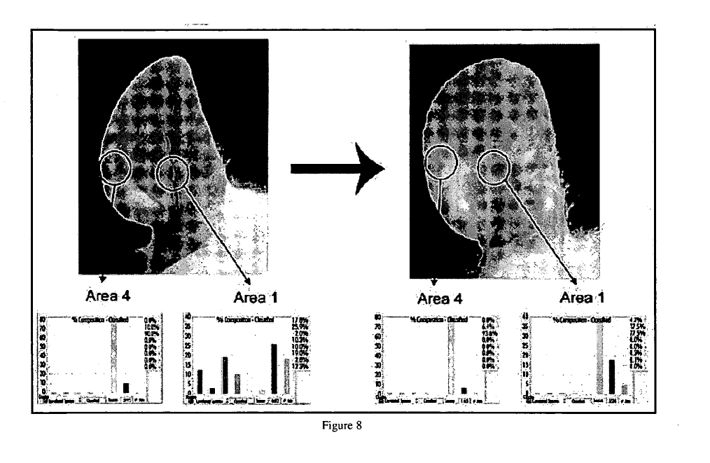

The results confirmed the hypothesis that the spectral histograms of Areas 5 and 9 closely resembled Areas 1 and 3. Additionally, it was found that Areas 6 and 7 displayed a “hybrid” spectral histogram between that of Areas 5, 9 and Area 8 (FIG. 7). This data led to the characterization of a unique vascular signature.

Overall, these findings led to the conclusion that the SCID mouse ear provides a model to establish a vascular hyperspectral signature spectrum as one moves away from the base of the ear, against gravity, towards to apex of the ear. This data also supports the conclusion that the MACRO-PARISS HSI system fiber-optic attachment provides reproducible vascular and non-vascular signatures. As the FOV moved from vascular to non-vascular areas, the acquired spectra changed in a step-wise predictable fashion.

### Example 3

### Removal the Blood Supply from a Vascular Field with Subsequent Spectral Acquisitions

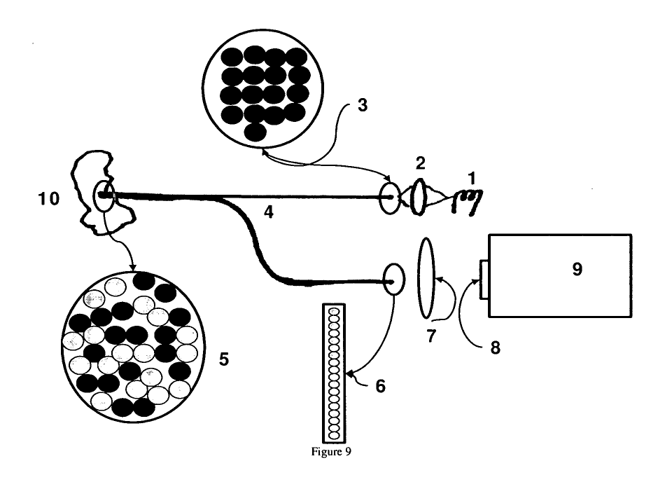

At the time of mouse sacrifice, vascular and non-vascular skin regions were imaged before and after stripping the vascular blood supply to the ear via a 1 cm surgical incision made at the base of the ear with subsequent drainage of the blood with gravity. It was hypothesized that removal of the blood supply would lead to vascular regions taking on a new non-vascular spectral signature. Additionally, it was expected that no change in spectral signatures in regions that were non-vascular prior to stripping the vascular blood supply. FIG. 8 illustrates vascular regions taking on non-vascular spectral signatures after the removal of their blood supply. This approach further supports the conclusion that the spectral signatures are indeed vascular and non-vascular.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A hyperspectral imaging system comprising: an electromagnetic energy source; coupled to a prism and reflector imaging spectroscopy system (PARISS) equipped with an imaging probe comprising a bundle of structured optical fibers capable of being used for a spatially resolved imaging.

2. The system of claim 1, wherein the electromagnetic energy source is a high intensity light source.

3. The system of claim 2, wherein the high intensity light source is a high intensity tungsten halogen lamp, or a Xenon lamp.

4. The system of claim 1, wherein the plurality of customized bundle of structured optical fibers that can be used as spatially resolved imaging probes are spatially discrete.

5. The system of claim 1, wherein the prism and reflector imaging spectroscopy system comprises an imaging spectrometer integrated with a first camera; and a second camera acting as an observed image camera.

6. The system of claim 5, wherein the first and second camera is a QICAM.

7. The system of claim 1, wherein the plurality of customized bundle of structured optical fibers that can be used as spatially resolved imaging probes comprise illumination probes and signal collection probes.

8. The system of claim 7, wherein the system collection probes are arrayed along a slit.

9. The system of claim 8, wherein the system collection probes collect images onto an entrance slit in the prism and reflector imaging spectroscopy system.

10. The system of claim 7, wherein the illumination probes and signal collection probes comprise no less than 16 illumination fibers and no less than 16 signal collection fibers.

11. The system of claim 1, wherein the remote fiber-optic probes are capable of being mapped to produce an image.

12. The system of claim 1, wherein the remote fiber-optic probes are distributed in a silicone cuff to be placed over an organ, a tissue or their combination.

13. A method of acquiring in-vivo hyperspectral image from a subject, comprising: selecting a field of view (FOV) of the subject; attaching a plurality of customized remote fiber-optic probes, wherein the customized remote fiber-optic probes are operably linked to a hyperspectral imaging system comprising: an electromagnetic energy source; coupled to a prism and reflector imaging spectroscopy system (PARISS) equipped with an imaging probe; illuminating the field of view using the hyperspectral imaging system; and collecting an in-vivo hyperspectral image.

14. The method of claim 13, whereby wherein the electromagnetic energy source is a high intensity light source.

15. The method of claim 14, whereby the high intensity light source is a high intensity tungsten halogen lamp.

16. The method of claim 13, whereby the plurality of customized bundle of structured optical fibers that can be used as a spatially resolved imaging probes are spatially discrete.

17. The method of claim 13, whereby the prism and reflector imaging spectroscopy system comprises an imaging spectrometer integrated with a first camera; and a second camera acting as an observed image camera.

18. The method of claim 17, whereby the first or second camera or both are QICAM

19. The method of claim 13, whereby the plurality of customized bundle of structured optical fibers that can be used as spatially resolved imaging probes comprise illumination probes and signal collection probes.

20. The method of claim 19, whereby the collection probes are arrayed along a slit.

21. The method of claim 20, whereby the collection probes collect images onto an entrance slit in the a prism and reflector imaging spectroscopy system.

22. The method of claim 19, whereby the illumination probes and signal collection probes comprise no less than 15 illumination fibers and no less than 16 signal collection fibers.

23. The method of claim 13, further comprising compiling a unique spectral signature of the field of view.

24. An image acquired by the method of claim 13.

25. The spectral signature of a field of view (FOV) compiled by the method of claim 23.

26. A method of monitoring neoplasia of a tissue in a subject, comprising the step of obtaining a hyperspectral image, according to the method of claim 13, of a field of view of an area sought to be monitored; and comparing the image to a standard.

27-37. (canceled)

38. The method of claim 26, further comprising comparing the spectral signature of the field of view with the spectral signature of the same field of view obtained from a subject exhibiting neoplasia.

39. The method of claim 26, further comprising comparing the spectral signature of the field of view with the spectral signature of the same field of view obtained from a subject not exhibiting neoplasia.

40. The method of claim 26, whereby the standard is a hyperspectral image of the tissue at a predetermined point.

41. The method of claim 40, whereby the predetermined point is time, course of treatment, dosage of a therapeutic agent or their combination.

42. A method of imaging a natural history or response to therapy of lesions of the skin, oropharynx, esophagus, bladder, or intra-abdominal lesions accessed through laparoscopy, comprising the step of obtaining a hyperspectral image, according to the method of claim 13, of a field of view of an area sought to be monitored in the lesions of the skin, oropharynx, esophagus, bladder, or intra-abdominal lesions accessed through laparoscopy; and comparing the image to a standard.

43-53. (canceled)

54. The method of claim 43, further comprising comparing the spectral signature of the field of view with the spectral signature of the same field of view obtained from a healthy subject.

55. The method of claim 42, whereby the standard is a hyperspectral image of the tissue at a predetermined point.

56. The method of claim 55, whereby the predetermined point is time, course of treatment, dosage of a therapeutic agent or their combination.

57. A library of spectral signatures of field of view obtained from the method of any one of claims 13, 26 and 42.
